(19) United States
(12) Patent Application Publication
(10) Pub. No.: US 2024/0018071 A1
(43) Pub. Date: Jan. 18, 2024

(54) MONOALKYLATION OF CYCLOPENTADIENE

(71) Applicant: ENTEGRIS, INC., Billerica, MA (US)

(72) Inventors: Scott A. Laneman, Vernon Hills, IL (US); Vagulejan Balasanthiran, Pennsburg, PA (US); Smita Patnaik, Warrington, PA (US); Victoria Weidner, Media, PA (US)

(21) Appl. No.: 18/220,704

(22) Filed: Jul. 11, 2023

Related U.S. Application Data
(60) Provisional application No. 63/389,696, filed on Jul. 15, 2022.

(51) Int. Cl. C07C 4/08

(52) U.S. Cl. CPC C07C 4/08

(57) ABSTRACT

The disclosure provides an improved method for preparing monoalkylated cyclopentadiene species in high yield and selectivity. In the process, either a solution of dicyclopentadiene magnesium or a cyclopentadiene magnesium halide is reacted with an alkylating agent in the presence of a modifying agent to provide the monoalkylated product. In the process of the disclosure, only a mono-alkylated species is produced with no detectible amount of dialkylated product observed.

### TECHNICAL FIELD

This disclosure generally relates to a process for preparing mono-alkylated cyclopentadiene compounds.

### BACKGROUND

Cyclopentadienes are useful as intermediates to many other useful organic compounds. Certain alkyl-substituted cyclopentadienes are useful as synthetic lubricants. (See, for example, U.S. Pat. Nos. 5,144,095 and 5,012,022.) Additionally, the cyclopentadiene structure can also be found in many of the so-called single site metallocene catalysts used to make polyolefins such as polyethylenes and polypropylenes. (See, for example, U.S. Pat. No. 7,579,415).

One inherent difficulty in the handling of cyclopentadiene is that it tends to dimerize via a Diels-Alder reaction. This dimerization proceeds at room temperature over a period of hours, but can be reversed by utilization of heating, which in some cases requires a cracking procedure. Additionally, in alkylation reactions utilizing a cyclopentadiene anion species, the formation of di- and tri-alkyl species can be encountered, which further complicates the synthetic regime by reducing yields and necessitating further separation and purification.

Thus, a need exists for improved methodology for the mono-alkylation of cyclopentadiene structures.

### SUMMARY

In summary, the disclosure provides an improved method for preparing monoalkylated cyclopentadiene species in high yield and selectivity. In the process, either a solution of dicyclopentadiene magnesium or a cyclopentadiene magnesium halide is reacted with an alkylating agent in the presence of a modifying agent to provide the monoalkylated product. In the process of the disclosure, only a mono-alkylated species is produced with no detectible amount of dialkylated product observed with measurement by gas chromatography.

### DETAILED DESCRIPTION

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The term “about” generally refers to a range of numbers that is considered equivalent to the recited value (e.g., having the same function or result). In many instances, the term “about” may include numbers that are rounded to the nearest significant figure.

Numerical ranges expressed using endpoints include all numbers subsumed within that range (e.g., 1 to 5 includes 1, 1.5, 2, 2.75, 3, 3.80, 4 and 5).

In a first aspect, the disclosure provides a process for preparing a compound of the Formula (I):

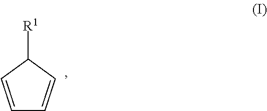

wherein R¹ is a straight or branched-chain C₁-C₈ alkyl group,
which comprises contacting a solution of a compound of the formula (A) or (B):

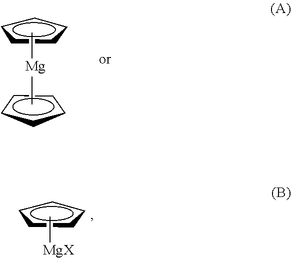

wherein X is halo,
with a modifying agent, followed by treatment with a compound of the formula R¹—X¹, wherein X¹ is halo or an alkyl or aromatic sulfonate.

In the process of the disclosure, the compound of formula (A) or (B) is desirably first dissolved or suspended in a solvent effective in at least partially dissolving the cyclopentadiene magnesium species or cyclopentadiene magnesium halide species, either alone or in combination with other solvents as described below. In one embodiment, tetrahydrofuran (THF) is utilized. Notably, attempts to dissolve (A) in dimethyl sulfoxide (DMSO) at room temperature resulted in an exothermic reaction which leads to a black/brown residue at room temperature. In the process of this disclosure, the exotherm of this reaction can be controlled at a lower temperature to result in the formation of the desired product (e.g., mono-alkylated cyclopentadiene). In the process of the disclosure, after the dissolution of the starting material (A) or (B), addition of a modifying agent, wherein the modifying agent is chosen so as to create a cyclopentadiene anion ring associated with the magnesium cation, followed by addition of a compound of the formula R¹—X¹, the desired product (i.e., compound of Formula I) is obtained in high yield with no detectible amount of dialkylated (or trialkylated) species observed by gas chromatography. In certain embodiments, the modifying agent is chosen from solvents such as dimethyl sulfoxide; dimethylacetamide; N-methyl-2-pyrrolidone; hexamethylphosphoramide; pyridine and its alkylated derivatives and alkylamino derivatives, an example of the latter being dimethylamino pyridine (DMAP); a crown ether; and combinations thereof. In one embodiment, the modifying agent is dimethyl sulfoxide. In some embodiments, the process may result in 1.0% or less, 0.75% or less, 0.50% or less, 0.25% or less 0.10% or less, 0.05% or less, or 0.01% or less of dialkylated (or trialkylated) products as measured by gas chromatography. In some embodiments, the conversion to the compound of Formula I may be 80% or greater, 82% or greater, 85% or greater, 87% or greater, 90% or greater, 92% or greater, or 95% or greater as measured by gas chromatography.

Groups of the formula —X¹ are suitable leaving groups such as halo, mesylate, tosylate, and the like. Exemplary compounds of the formula R¹—X¹ include methyl bromide, methyl iodide, ethyl bromide, ethyl iodide, isopropyl bromide, isopropyl iodide, ethyl tosylate, isopropyl tosylate, ethyl mesylate, isopropyl mesylate, and the like.

Exemplary solvents useful for the purpose of dissolving/suspending the compound of formula (A) or (B) include solvents such as tetrahydrofuran, diethyl ether, toluene, and the like, with the only consideration being the desirability that the compound of formula (A) or (B) is at least partially soluble in the solvent.

As used herein, the term “crown ether” denotes those cyclic compounds containing several ether groups. Exemplary crown ethers include cyclic oligomers of ethylene oxide, including nitrogen-containing macrocycles. Examples include 12-crown-4, 15-crown-5, 18-crown-6, dibenzo-18-crown-6, and aza-crown. Numerous crown ethers are available commercially from Sigma Aldrich.

In certain embodiments, the modifying agent is present in an amount of at least about 3 molar equivalents, based on the amount of the compound of formula (A) or (B) present. In other embodiments, the modifying agent is present in an amount of 3 molar equivalents to about 50 molar equivalents, based on the amount of the compound of formula (A) or (B) present, and in other embodiments, the modifying agent is present in an amount of about 6 to about 15 molar equivalents, based on the amount of the compound of formula (A) or (B) present.

As noted above, the alkylating agent is a compound of the formula R¹-X, wherein R¹ is a straight or branched-chain C₁-C₈ alkyl group, and X is halo, for example bromo or iodo. In certain embodiments, R¹ is chosen from methyl, ethyl, n-propyl, n-butyl, sec-butyl and the like. In certain embodiments, R¹ is a branched chain group such as isopropyl. Surprisingly, the result of the reaction is mono-alkylation of the cyclopentadiene ring, with no dialkylated species detected by gas chromatography.

In various embodiments, R¹ is chosen from methyl, ethyl, and isopropyl.

### EXAMPLES

### Example 1 —Synthesis of Bis(η⁵-Cyclopentadienlyl)Magnesium(II)-Cp₂Mg

Freshly cracked cyclopentadiene (50 g, 0.76 mol) was slowly added at room temperature to 0.7M di-ⁿbultylmagnesium in hexanes [350 mL, di-ⁿbultylmagnesium or di-^{sec}bultylmagnesium or ⁿbultly' ^{sec}bultylmagnesium in hexanes or heptanes can be used] in a 1 L Schlenk flask under nitrogen with stirring. The temperature during the addition was maintained using isopropanol/dry ice bath at 22±3° C. After complete addition, the reaction stirred at room temperature for 6 hours, then cooled to 10° C., which caused the product to settle in the flask. The mother liquor was removed by using a cannula. All volatiles were removed under vacuum to produce 44.2 g of Cp₂Mg with 82% yield. ¹H NMR (C₆D6): 6.00 ppm (s, 12H, Cp-H); ¹³C NMR (C₆D₆): 107.6-107.8 ppm (bm, Cp-CH)

### Examples 2 Through 5 —Synthesis of Isopropyl-CpH (iPrCpH)

As shown in Table 1 below, ⁱPrCpH was synthesized under four conditions. For examples 2 and 3, a 250 mL Schlenk flask was charged Cp₂Mg in the amount shown in Table 1 at room temperature under nitrogen followed by THF (46.7 g) with stirring. Cp₂Mg was dissolved completely. ⁱPrBr (isopropyl bromide) in the amount shown in Table 1 was added slowly with stirring to the Cp₂Mg solution. The resulting mixture was stirred for the time and temperature shown in Table 1 then quenched with 5% HCl solution (50 mL). The organic phase was separated, and gas chromatography (GC) analysis showed the percent conversion to ⁱPrCpH.

For examples 4 and 5, a 250 mL Schlenk flask was charged Cp₂Mg in the amount shown in Table 1 at room temperature under nitrogen followed by THF (46.7 g) with stirring. Cp₂Mg was dissolved completely and anhydrous DMSO, in the amount shown in Table 1, was added slowly with stirring to the Cp₂Mg solution. The resulting mixture/slurry was stirred for 30 min or until a consistent free flowing liquid formed. ⁱPrBr (isopropyl bromide) in the amount shown in Table 1 was added slowly with stirring to the Cp₂Mg/DMSO slurry. The resulting mixture was stirred for the time and temperature shown in Table 1 then quenched with 5% HCl solution (50 mL). The organic phase was separated, and GC analysis showed the percent conversion to ⁱPrCpH. No detectible amount of dialkylated (or trialkylated) species were observed by gas chromatography.

The results in Table 1 show that the addition of DMSO as a modifying agent in Examples 4 and 5 led to higher conversions of ⁱPrCpH (greater than 90%) than examples 2 and 3 where a modifying agent such as DMSO was not added.

TABLE 1
Results for the synthesis of ⁱPrCpH

| | | | | | ⁱPrBr/ | | | DMSO/ | | | | |
|---|---|---|---|---|---|---|---|---|---|---|---|---|
| | Cp₂Mg | Cp₂Mg | ⁱPrBr | ⁱPrBr | Cp₂Mg | THF | DMSO | Cp₂Mg | Time | Temp | % | % |
| Ex | (g) | (mmol) | (g) | (mmol) | ratio | (g) | (g) | ratio | (hrs) | (° C.) | ⁱPrCpH | Cp |
| 2 | 1.0 | 6.47 | 2.5 | 20.33 | 3.14 | 10 | 0 | 0.0 | 24 | RT | 67.7 | 32.3 |
| 3 | 5.0 | 32.36 | 10 | 81.31 | 2.51 | 50 | 0 | 0.0 | 24 | 50° C. | 65.3 | 34.7 |
| 4 | 1.0 | 6.47 | 1.7 | 13.82 | 2.14 | 25 | 10 | 19.8 | 1 | RT | 92.8 | 7.2 |
| 5 | 0.5 | 3.24 | 0.85 | 6.91 | 2.14 | 11 | 8 | 31.6 | 1 | RT | 92 | 3.59 |

### Examples 6 Through 7—Synthesis of Ethyl-Cp (EtCpH)

As shown in Table 2 below, EtCpH was synthesized under two conditions. For Example 6, a 250 mL Schlenk flask was charged Cp₂Mg at room temperature under nitrogen followed by THF with stirring. Cp₂Mg was dissolved completely, and anhydrous DMSO was added slowly with stirring to the Cp₂Mg solution. The resulting mixture/slurry was stirred for 30 min or until a consistent free flowing liquid formed. Ethyl bromide (EtBr) was added slowly with stirring to the Cp₂Mg/DMSO slurry. The resulting mixture was stirred for 1 hour at room temperature then quenched with 5% HCl solution (50 mL). The organic phase was separated, and gas chromatography (GC) analysis showed the percent conversion to EtCpH. No detectible amount of dialkylated (or trialkylated) species were observed by gas chromatography (GC) analysis.

For Example 7, a 250 mL Schlenk flask was charged Cp₂Mg at room temperature under nitrogen followed by THF with stirring. Cp₂Mg was dissolved completely. Ethyl bromide (EtBr) was added slowly with stirring to the Cp₂Mg solution. The resulting mixture was stirred for 1 hour at room temperature then quenched with 5% HCl solution (50 mL). The organic phase was separated, and gas chromatography (GC) analysis showed the percent conversion to EtCpH.

The results in Table 1 show that the addition of DMSO as a modifying agent in Example 6 lead to higher conversions of EtCpH (greater than 90%) than Example 7 where a modifying agent such as DMSO was not added.

TABLE 2
Selected results for the synthesis of Ethyl-Cyclopentadiene

| | | | | | EtBr/ | | | DMSO/ | | | | |
|---|---|---|---|---|---|---|---|---|---|---|---|---|
| | Cp₂Mg | Cp₂Mg | EtBr | EtBr | Cp₂Mg | THF | DMSO | Cp₂Mg | Time | Temp | % | % |
| Ex | (g) | (mmol) | (g) | (mmol) | ratio | (g) | (g) | ratio | (hrs) | (° C.) | EtCpH | Cp |
| 6 | 1.0 | 6.47 | 1.8 | 16.52 | 2.55 | 15 | 7 | 13.8 | 1 | RT | 91.4 | 8.6 |
| 7 | 1.0 | 6.47 | 1.8 | 16.52 | 2.55 | 15 | 0 | 0.0 | 1 | RT | 58.13 | 40.4 |

### ASPECTS

In a first aspect, the disclosure provides a process for preparing a compound of the Formula (I):

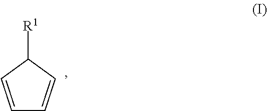

wherein R¹ is a straight or branched-chain C₁-C₈ alkyl group,
which comprises contacting a solution of a compound of the formula (A) or (B):

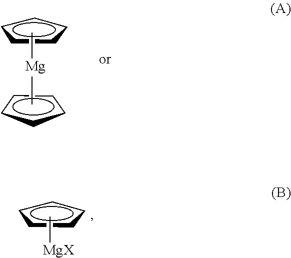

wherein X is halo,
with a modifying agent, followed by treatment with a compound of the formula R¹—X¹, wherein X¹ is halo or an alkyl or aromatic sulfonate.

In a second aspect, the disclosure provides the process of the first aspect, wherein the modifying agent is chosen from the group consisting of dimethyl sulfoxide; dimethylacetamide; N-methyl-2-pyrrolidone; hexamethylphosphoramide; pyridine and its alkylated derivatives, and alkylamino derivatives, such as dimethylamino pyridine (DMAP); a crown ether; and combinations thereof.

In a third aspect, the disclosure provides the process of the first or second aspect, wherein R¹ is isopropyl.

In a fourth aspect, the disclosure provides the process of claim 1, wherein the alkyl or aryl sulfonate is a mesylate or a tosylate.

In a fifth aspect, the disclosure provides the process of any one of the first through fourth aspects, wherein the modifying agent is present in an amount of at least about 3 molar equivalents, based on the amount of the compound of formula (A) or (B) present.

In a sixth aspect, the disclosure provides the process of any one of the first through the fourth aspects, wherein the modifying agent is present in an amount of 3 molar equivalents to about 50 molar equivalents, based on the amount of the compound of formula (A) or (B) present.

In a seventh aspect, the disclosure provides the process of any one of the first through the fourth aspects, wherein the modifying agent is present in an amount of about 6 to about 15 molar equivalents, based on the amount of the compound of formula (A) or (B) present.

In an eighth aspect, the disclosure provides the process of any one of the first through the seventh aspects, wherein the modifying agent is dimethyl sulfoxide.

In a ninth aspect, the disclosure provides a process for preparing a compound of the Formula (I):

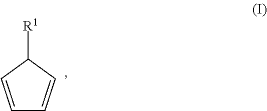

wherein R¹ is a straight or branched-chain C₁-C₈ alkyl group,
which comprises contacting a solution of a compound of the formula (A):

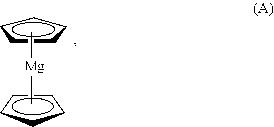

with a modifying agent, followed by treatment with a compound of the formula R¹—X¹, wherein X¹ is halo or an alkyl or aromatic sulfonate.

In a tenth aspect, the disclosure provides the process of the ninth aspect, wherein the modifying agent is dimethyl sulfoxide.

In an eleventh aspect, the disclosure provides the process of the ninth or tenth aspect, wherein R¹ is chosen from methyl, ethyl, isopropyl, n-butyl, or sec-butyl.

In a twelfth aspect, the disclosure provides the process of the ninth, tenth, or eleventh aspect, wherein R¹ is isopropyl.

In a thirteenth aspect, the disclosure provides the process of the ninth, tenth, or eleventh aspect, wherein R¹ is ethyl.

In a fourteenth aspect, the disclosure provides a process for preparing a compound of the Formula (I):

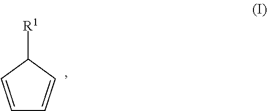

wherein R¹ is a straight or branched-chain C₁-C₈ alkyl group,
which comprises contacting a solution of a compound of the formula (B):

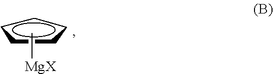

wherein X is halo,
with a modifying agent, followed by treatment with a compound of the formula R¹—X¹, wherein X¹ is halo or an alkyl or aromatic sulfonate.

In a fifteenth aspect, the disclosure provides the process of the fourteenth aspect, wherein the modifying agent is dimethyl sulfoxide.

In a sixteenth aspect, the disclosure provides the process of the fourteenth or fifteenth aspect, wherein R¹ is chosen from methyl, ethyl, and isopropyl.

In a seventeenth aspect, the disclosure provides the process of the fourteenth, fifteenth, or sixteenth aspects, wherein R¹ is isopropyl.

In an eighteenth aspect, the disclosure provides the process of any preceding aspect, wherein 1.0% or less, 0.75% or less, 0.50% or less, 0.25% or less 0.10% or less, 0.05% or less, or 0.01% or less of dialkylated compounds are formed as measured by gas chromatography.

In a nineteenth aspect, the disclosure provides the process of any preceding aspect, wherein conversion to the compound of Formula (I) may be 80% or greater, 82% or greater, 85% or greater, 87% or greater, 90% or greater, 92% or greater, or 95% or greater as measured by gas chromatography.

Having thus described several illustrative embodiments of the present disclosure, those of skill in the art will readily appreciate that yet other embodiments may be made and used within the scope of the claims hereto attached. Numerous advantages of the disclosure covered by this document have been set forth in the foregoing description. It will be understood, however, that this disclosure is, in many respects, only illustrative. The disclosure's scope is, of course, defined in the language in which the appended claims are expressed.

What is claimed is:

1. A process for preparing a compound of the Formula (I): 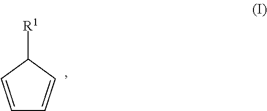
wherein R¹ is a straight or branched-chain C₁-C₈ alkyl group, the process comprising:
contacting a solution of a compound of the formula (A): 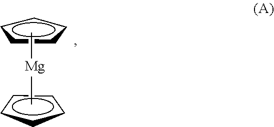
with a modifying agent to form a mixture; and
treating the mixture with a compound of the formula R¹—X¹, wherein X¹ is halo or an alkyl or aromatic sulfonate, thereby forming a compound of the Formula (I).

2. The process of claim 1, wherein 1.0% or less of dialkylated compounds are formed as measured by gas chromatography.

3. The process of claim 1, wherein conversion to the compound of Formula (I) may be 80% or greater.

4. The process of claim 1, wherein the modifying agent is chosen from the group consisting of dimethyl sulfoxide; dimethylacetamide; N-methyl-2-pyrrolidone;
hexamethylphosphoramide; pyridine and its alkylated derivatives and alkylamino derivatives; a crown ether; and combinations thereof.

5. The process of claim 4, wherein the modifying agent is dimethyl sulfoxide.

6. The process of claim 1, wherein R¹ is chosen from methyl, ethyl, isopropyl, n-butyl, or sec-butyl.

7. The process of claim 6, wherein R¹ is isopropyl.

8. The process of claim 6, wherein R¹ is ethyl.

9. The process of claim 1, wherein the alkyl or aryl sulfonate is a mesylate or a tosylate.

10. The process of claim 1, wherein the modifying agent is present in an amount of at least about 3 molar equivalents, based on the amount of the compound of formula (A).

11. The process of claim 10, wherein the modifying agent is present in an amount of about 3 molar equivalents to about 50 molar equivalents, based on the amount of the compound of formula (A) present.

12. The process of claim 10, wherein the modifying agent is present in an amount of about 6 to about 15 molar equivalents, based on the amount of the compound of formula (A).

13. A process for preparing a compound of the Formula (I): 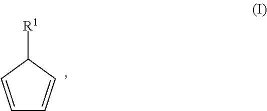
wherein R¹ is a straight or branched-chain C₁-C₈ alkyl group, the process comprising:
contacting a solution of a compound of the formula (B): 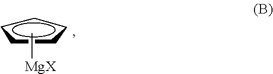
wherein X is halo, with a modifying agent to form a mixture; and
treating the mixture with a compound of the formula R¹—X¹, wherein X¹ is halo or an alkyl or aromatic sulfonate.

14. The process of claim 13, wherein 1.0% or less of dialkylated compounds are formed as measured by gas chromatography.

15. The process of claim 13, wherein conversion to the compound of Formula (I) may be 80% or greater.

16. The process of claim 13, wherein the modifying agent is chosen from the group consisting of dimethyl sulfoxide; dimethylacetamide; N-methyl-2-pyrrolidone;
hexamethylphosphoramide; pyridine and its alkylated derivatives and alkylamino derivatives; a crown ether; and combinations thereof.

17. The process of claim 13, wherein the modifying agent is dimethyl sulfoxide.

18. The process of claims 13, wherein R¹ is chosen from methyl, ethyl, isopropyl, n-butyl, or sec-butyl.

19. The process of claim 18, wherein R¹ is isopropyl.

20. The process of claim 18, wherein R¹ is ethyl.

21. The process of claim 13, wherein the alkyl or aryl sulfonate is a mesylate or a tosylate.

22. The process of claim 13, wherein the modifying agent is present in an amount of at least about 3 molar equivalents, based on the amount of the compound of formula (A).

23. The process of claim 22, wherein the modifying agent is present in an amount of about 3 molar equivalents to about 50 molar equivalents, based on the amount of the compound of formula (A) present.

24. The process of claim 22, wherein the modifying agent is present in an amount of about 6 to about 15 molar equivalents, based on the amount of the compound of formula (A).
